(19) United States
(12) Patent Application Publication
(10) Pub. No.: US 2025/0050110 A1
(43) Pub. Date: Feb. 13, 2025

(54) METHODS AND DEVICES FOR CONTROLLED DELIVERY OF NEURAL STIMULATION

(71) Applicant: Saluda Medical Pty Ltd, Macquarie Park, New South Wales (AU)

(72) Inventors: Matthew Marlon Williams, Macquarie Park (AU); Daniel John Parker, Macquarie Park (AU); Samuel Nicholas Gilbert, Macquarie Park (AU); Dean Michael Karantonis, Macquarie Park (AU); Milan Obradovic, Macquarie Park (AU); Peter Scott Vallack Single, Macquarie Park (AU)

(21) Appl. No.: 18/722,901

(22) PCT Filed: Dec. 23, 2022
(86) PCT No.: PCT/AU2022/051583

§ 371 Date: Jun. 21, 2024

(30) Foreign Application Priority Data
Dec. 23, 2021 (AU) 2021904239

(51) Int. Cl. A61N 1/36; G16H 20/40; G16H 40/63

(52) U.S. Cl. CPC A61N 1/36139; A61N 1/3615; G16H 20/40; G16H 40/63

(57) ABSTRACT

Disclosed are methods and devices for analysing signal windows captured subsequent to delivered neural stimuli and for providing a quality score for each signal window. The quality score is indicative of how closely the captured signal window resembles, or how likely that the captured signal window contains, an ECAP. The quality score may be provided to a process supervising a feedback loop of the closed-loop neural stimulation device delivering the stimuli and capturing the signal windows to ensure the delivered stimuli are appropriate. For example, if the score falls below a predetermined threshold, the supervisor may take a mitigation action to prevent inappropriate adjustment to the intensity of the delivered stimuli by the feedback loop, such as suspending operation of the feedback loop.

### TECHNICAL FIELD

The present invention relates to closed-loop neural stimulation devices and in particular to methods of supervising the feedback control of neural stimulation carried out by such devices.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation system applies an electrical pulse (stimulus) to neural tissue (fibres, or neurons) in order to generate a therapeutic effect. In general, the electrical stimulus generated by a neuromodulation system evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a system typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. The fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres. When recruitment is too large, Aβ fibres produce uncomfortable sensations. Stimulation at high intensity may even recruit Aδ fibres, which are sensory nerve fibres associated with acute pain, cold and pressure sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Another control problem facing neuromodulation systems of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable systems, increased power consumption results in more frequent charging and, given that batteries only permit a limited number of charging cycles, ultimately this reduces the implanted lifetime of the device.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the sensed signal as crosstalk of that magnitude. Moreover, stimulus generally results in electrode artefact, which manifests in the sensed signal as a decaying output of the order of several millivolts after the end of the stimulus. As the neural response can be contemporaneous with the stimulus crosstalk and/or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Evoked neural responses are less difficult to measure when they appear later in time than the artefact, or when the signal-to-noise ratio is sufficiently high. The artefact is often restricted to a time of 1-2 ms after the stimulus and so, provided the neural response is measured after this time window, a neural response measurement can be more easily obtained. This is the case in surgical monitoring where there are large distances (e.g. more than 12 cm for nerves conducting at 60 ms⁻¹) between the stimulus and measurement electrodes so that the propagation time from the stimulus site to the measurement electrodes exceeds 2 ms, which is longer than the typical duration of stimulus artefact.

However, to characterize the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity. In such situations the measurement process must overcome artefact directly.

Regardless of how well-designed the ECAP measurement process may be, circumstances will still arise in which ECAPs become so difficult to measure that effective closed-loop control of neural stimulation becomes unfeasible. One example of such a circumstance is wireless charging of a battery of the implanted device, which can create such high levels of noise in the sensed signals containing the ECAPs that the ECAPs become effectively undetectable. However, a conventional ECAP detector will be unaware of this situation and will still return values for the ECAP amplitude after each stimulus, even though these values may be highly corrupted by charging-induced noise. Such corrupted values can lead to improper control of stimulus intensity.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are methods and devices for analysing signal windows captured subsequent to delivered neural stimuli and for providing a quality score for each signal window. The quality score is indicative of how closely the captured signal window resembles, or how likely that the captured signal window contains, an ECAP. The quality score may be provided to a process supervising a feedback loop of the closed-loop neural stimulation device delivering the stimuli and capturing the signal windows to ensure the delivered stimuli are appropriate. For example, if the score falls below a predetermined threshold, the supervisor may take a mitigation action to prevent inappropriate adjustment to the intensity of the delivered stimuli by the feedback loop, such as suspending operation of the feedback loop.

According to a first aspect of the present technology, there is provided an implantable device for controllably delivering neural stimuli, the device comprising: . . .
a plurality of electrodes including one or more stimulus electrodes and one or more sense electrodes;
a stimulus source configured to provide neural stimuli to be delivered via the one or more stimulus electrodes to a neural pathway of a patient in order to evoke a neural response from the neural pathway;
measurement circuitry configured to capture signal windows sensed on the neural pathway via the one or more sense electrodes subsequent to respective neural stimuli; and
a control unit configured to:
   control the stimulus source to provide a neural stimulus according to a stimulus intensity parameter;
   measure an intensity of an evoked neural response in the captured signal window subsequent to the provided neural stimulus;
   compute a feedback variable from the measured intensity of the evoked neural response; and
   implement a feedback controller configured to use the computed feedback variable to control the stimulus intensity parameter so as to maintain the feedback variable at a target value;
   compute a quality score from the captured signal window;
   determine whether the quality score meets one or more criteria indicative of satisfactory quality; and
   take mitigation action based on the determining.

According to a second aspect of the present technology, there is provided an automated method of controllably delivering neural stimuli to a neural pathway of a patient, the method comprising:
delivering a neural stimulus to the neural pathway of the patient in order to evoke a neural response from the neural pathway, the neural stimulus being delivered according to a stimulus intensity parameter;
capturing a signal window sensed on the neural pathway subsequent to the delivered neural stimulus;
measuring an intensity of a neural response evoked by the delivered neural stimulus in the captured signal window,
computing, from the measured intensity of the evoked neural response, a feedback variable; and
completing a feedback loop by using the computed feedback variable to control the stimulus intensity parameter so as to maintain the feedback variable at a target value; and
computing a quality score from the captured signal window;
determining whether the quality score meets one or more criteria indicative of satisfactory quality; and
taking mitigation action based on the determining.

In some embodiments, the control unit may be configured to compute the quality score by computing a difference between the captured signal window and a predetermined noise model for the captured signal windows. The control unit may be configured to compute the difference by: counting a number of outliers in the signal window, wherein an outlier is a sample that departs from the predetermined noise model; and computing a metric that quantifies a ratio of outliers present in the signal window relative to an expected ratio of outliers in a signal window that obeys the predetermined noise model. The control unit may be configured to apply a sigmoid function to the metric. The outlier may be a sample that differs from the mean of the predetermined noise model by more than n times the standard deviation of the predetermined noise model, wherein n is a small integer. The predetermined noise model may be a Gaussian model having a mean and a standard deviation. The control unit may be further configured to estimate the mean and the standard deviation from signal windows captured without preceding neural stimuli. The control unit may be further configured to remove stimulus artefact from the captured signal window before computing the difference.

In some embodiments, the control unit may be configured to compute the quality score by: computing a normalised correlation function representing a resemblance of the captured signal window to a correlation template.

In some embodiments the control unit may be configured to compute the quality score by: computing a plurality of component correlation functions, each component correlation function representing a resemblance of the captured signal window to a portion of a correlation template; and combining the component correlation functions into a combined correlation function. A peak value of the combined correlation function may be the quality score.

In some embodiments the control unit may be configured to compute the quality score by: fitting, for each of a plurality of combination models, the combination model to the captured signal window; computing a plurality of goodness-of-fit metrics indicative of the quality of the model fit of the respective combination models to the captured signal window; and computing the quality score from the plurality goodness-of-fit metrics for the respective combination models. Each combination model may comprise one or more component models. Computing the quality score may comprise computing a difference between a goodness-of-fit metric for a combination model comprising an ECAP component model and an artefact component model, and a goodness-of-fit metric for a combination model comprising an artefact component model alone. Computing the quality score may comprise computing a difference between a largest goodness-of-fit metric of the plurality of the goodness-of-fit metrics, and a goodness-of-fit metric for a most complex of the combination models.

In some embodiments the control unit may be configured to determine whether the quality score meets one or more criteria indicative of satisfactory quality by comparing the quality score with a threshold.

In some embodiments the control unit may be configured to take mitigation action by suspending the operation of the feedback controller. Computing the quality score may comprise computing a difference between the captured signal window and a predetermined noise model for the captured signal windows. Computing the quality score may comprise computing a normalised correlation function representing a resemblance of the captured signal window to a correlation template. Computing the quality score may comprise: computing a plurality of component correlation functions, each component correlation function representing a resemblance of the captured signal window to a portion of a correlation template; and combining the component correlation functions into a combined correlation function. Computing the quality score may comprise: fitting, for each of a plurality of combination models, the combination model to the captured signal window; computing a plurality of goodness-of-fit metrics indicative of the quality of the model fit of the respective combination models to the captured signal window; and computing the quality score from the plurality goodness-of-fit metrics for the respective combination models. Determining whether the quality score meets one or more criteria indicative of satisfactory quality may comprise comparing the quality score with a threshold. Taking mitigation action may comprise suspending the operation of the feedback controller.

According to a third aspect of the present technology, there is provided a closed-loop neural stimulation device for controllably delivering neural stimuli, the device comprising:
a feedback controller configured to use one or more controller parameters to control a stimulus intensity parameter so as to maintain a neural response intensity measured from a captured signal window at a target value; and
a processor configured to:
   compute a quality score from the captured signal window;
   determine whether the quality score meets one or more criteria indicative of satisfactory quality; and
   take mitigation action based on the determining.

According to a fourth aspect of the present technology, there is provided a neural stimulation system comprising:
an implantable device for controllably delivering neural stimuli, the device comprising:
   a plurality of electrodes including one or more stimulus electrodes and one or more sense electrodes;
   a stimulus source configured to provide neural stimuli to be delivered via the one or more stimulus electrodes to a neural pathway of a patient in order to evoke a neural response from the neural pathway; and
   measurement circuitry configured to capture signal windows sensed on the neural pathway via the one or more sense electrodes subsequent to respective neural stimuli; and
   a control unit configured to control the stimulus source to provide each neural stimulus according to a stimulus intensity parameter;
a processor configured to:
   instruct the control unit to control the stimulus source to provide a neural stimulus according to a stimulus intensity parameter;
   measure an intensity of an evoked neural response in the captured signal window subsequent to the provided neural stimulus;
   compute a feedback variable from the measured intensity of the evoked neural response;
   implement a feedback controller configured to use the computed feedback variable to control the stimulus intensity parameter so as to maintain the feedback variable at a target value;
   compute a quality score from the captured signal window;
   determine whether the quality score meets one or more criteria indicative of satisfactory quality; and
   take mitigation action based on the determining.

In some embodiments the processor is configured to compute the quality score by computing a difference between the captured signal window and a predetermined noise model for the captured signal windows.

In some embodiments the processor is configured to compute the quality score by computing a normalised correlation function representing a resemblance of the captured signal window to a correlation template.

In some embodiments the processor is configured to compute the quality score by: computing a plurality of component correlation functions, each component correlation function representing a resemblance of the captured signal window to a portion of a correlation template; and combining the component correlation functions into a combined correlation function.

In some embodiments the processor is configured to compute the quality score by: fitting, for each of a plurality of combination models, the combination model to the captured signal window; computing a plurality of goodness-of-fit metrics indicative of the quality of the model fit of the respective combination models to the captured signal window; and computing the quality score from the plurality goodness-of-fit metrics for the respective combination models.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation system, according to one implementation of the present technology;

FIG. 6 illustrates the typical form of an electrically evoked compound action potential (ECAP) of a healthy subject;

FIG. 7 is a block diagram of a neuromodulation therapy system including the implanted stimulator of FIG. 1 according to one implementation of the present technology;

FIG. 8 is a flow chart illustrating a method carried out by the controller as one implementation of a supervisor according to the present technology;

FIG. 9 is a flow chart illustrating a method of computing a quality score using regression and component models, as may be used by the supervisor to implement a step of the method of FIG. 8 according to one implementation of the present technology;

FIG. 10 contains a graph illustrating the five basis functions in a CPE artefact model; and

FIG. 11 is a flow chart illustrating a method carried out by the supervisor according to some implementations of the present technology.

### DETAILED DESCRIPTION OF THE PRESENT TECHNOLOGY

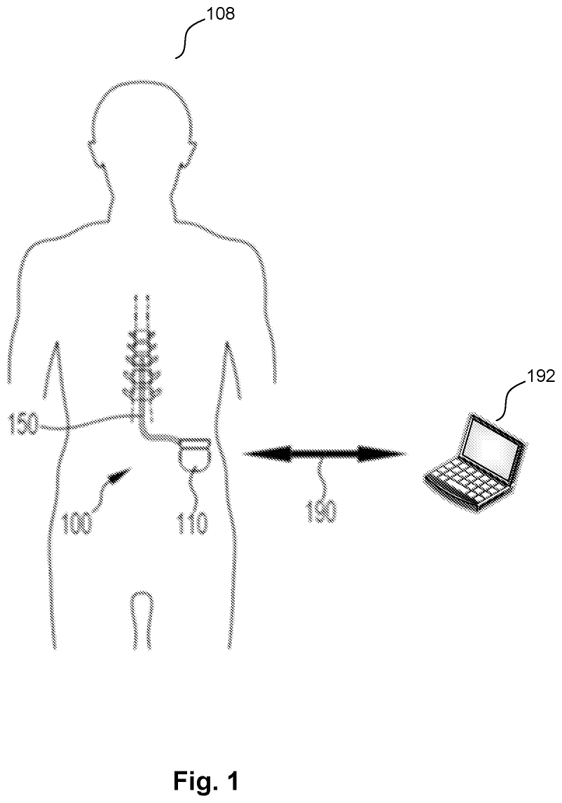

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

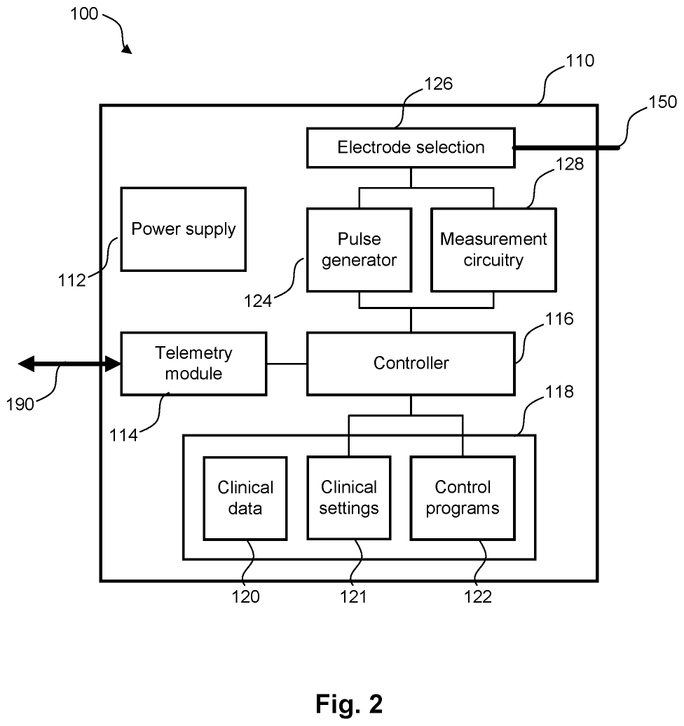

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

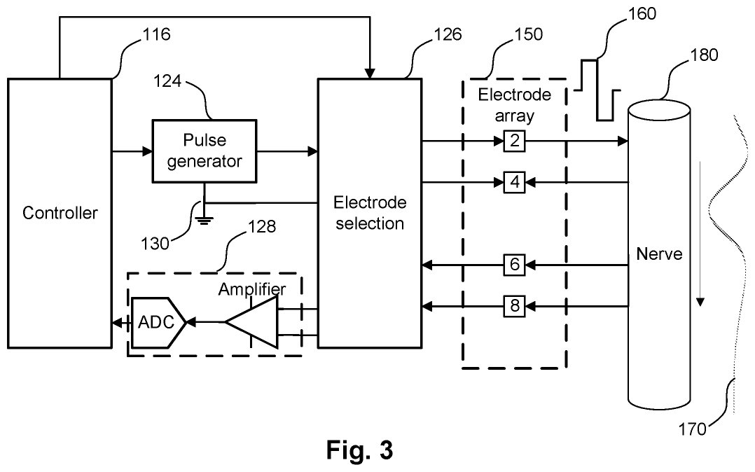

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes. The set of stimulus electrodes and return electrodes is referred to as the stimulus electrode configuration. Electrode selection module 126 is illustrated as connecting to a ground 130 of the pulse generator 124 to enable stimulus current return via the return electrode 4. However, other connections for charge recovery may be used in other implementations.

Delivery of an appropriate stimulus from electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

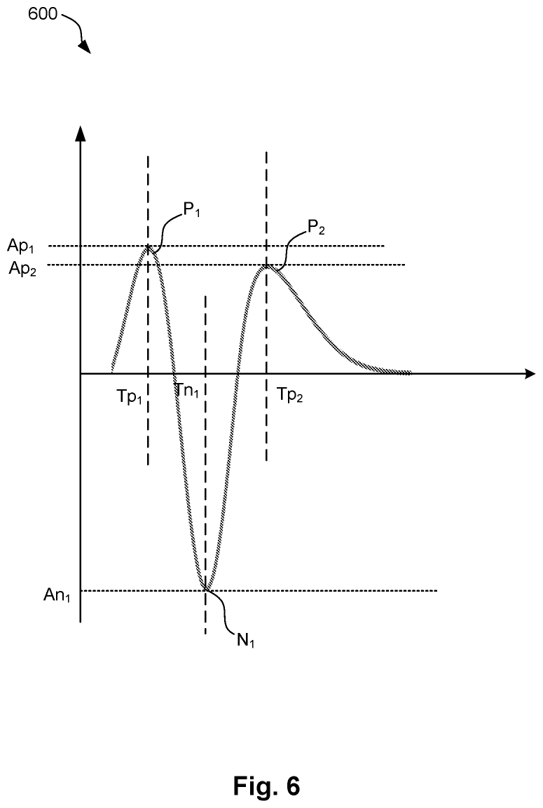

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristic comprises a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

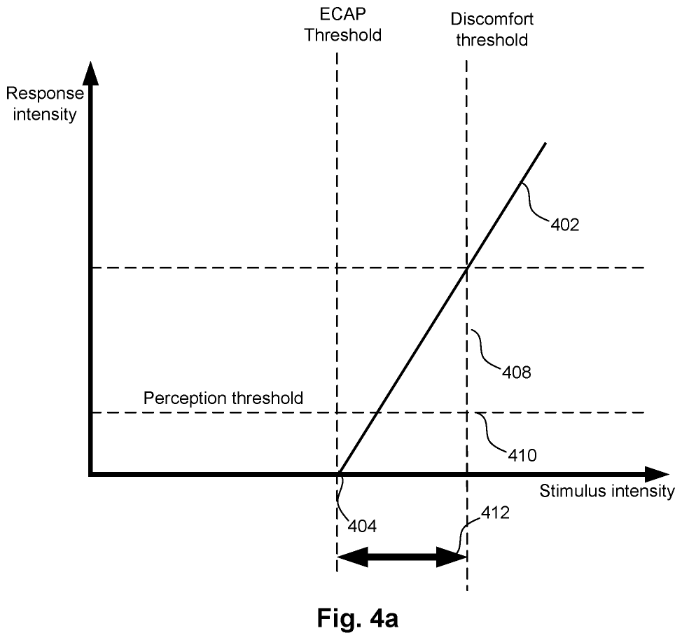

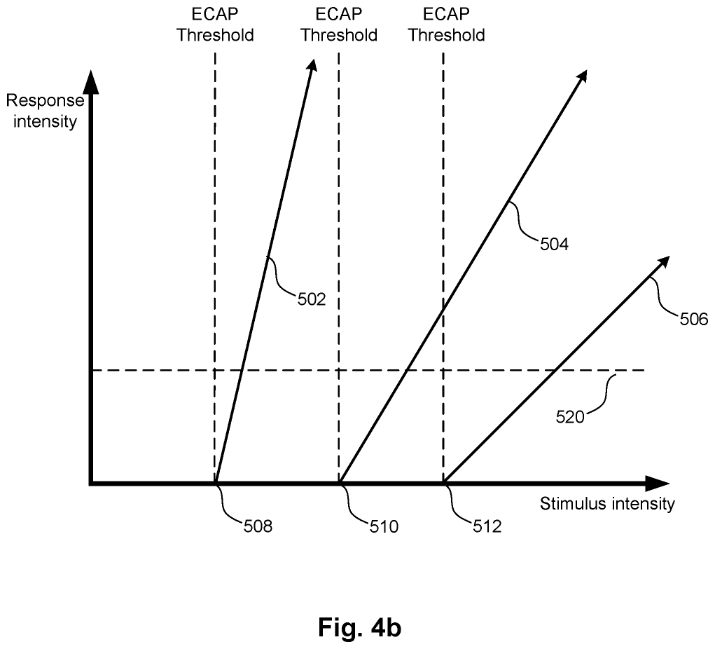

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, T is the ECAP threshold and S is the slope of the activation plot (referred to herein as the patient sensitivity). The slope S and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

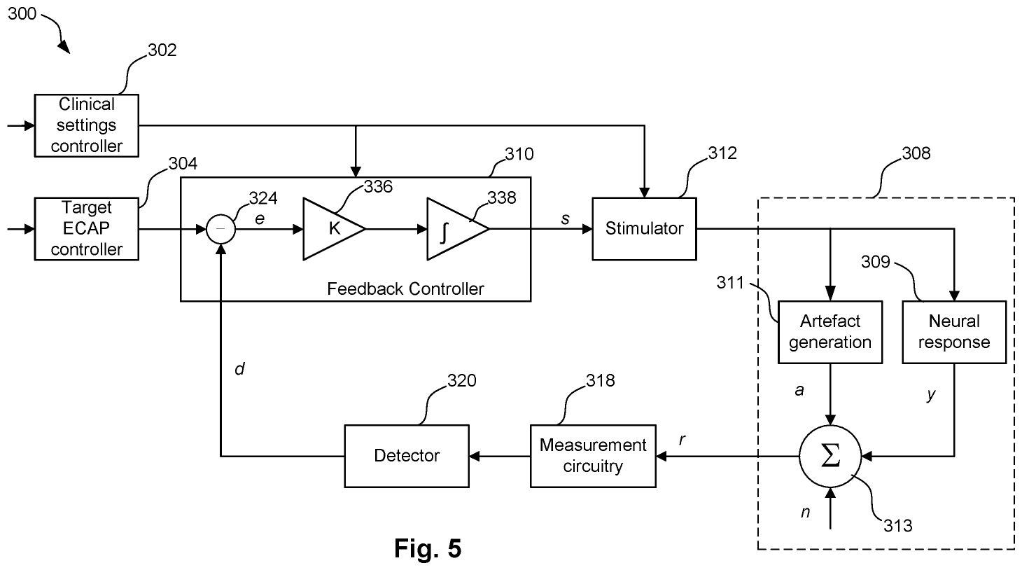

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and noise), and samples the amplified sensed signal r to capture a “signal window” comprising a predetermined number of samples of the amplified sensed signal r. The ECAP detector 320 processes the signal window and outputs a measured neural response intensity d. In one implementation, the neural response intensity comprises a peak-to-peak ECAP amplitude. The measured response intensity d is input into the feedback controller 310. The feedback controller 310 comprises a comparator 324 that compares the measured response intensity d to the target ECAP amplitude as set by the target controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be computed by the feedback controller 310 as

$$s = \int Kedt \qquad ( 2 )$$

where K is the gain of the gain element 336 (the controller gain). This relation may also be represented as

δs=Ke
where δs is an adjustment to the current stimulus intensity parameter s.

A target ECAP amplitude is input to the feedback controller 310 via the target controller 304. In one embodiment, the target controller 304 provides an indication of a specific target ECAP amplitude. In another embodiment, the target controller 304 provides an indication to increase or to decrease the present target ECAP amplitude. The target controller 304 may comprise an input into the CLNS system 300, via which the patient or clinician can input a target ECAP amplitude, or indication thereof. The target controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

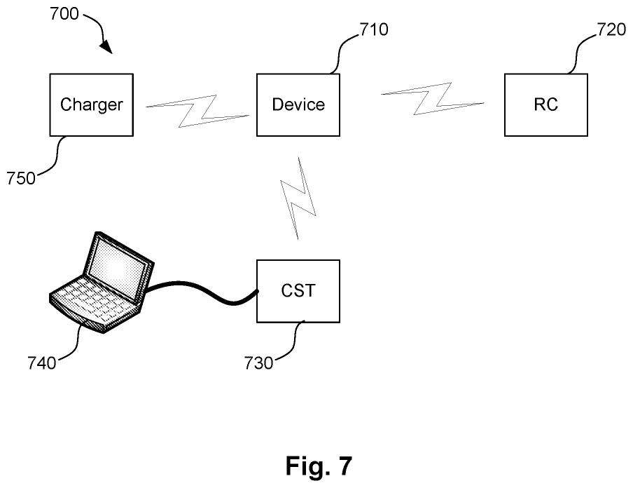

FIG. 7 is a block diagram of a neuromodulation system 700. The neuromodulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### Supervisor

The controller 116 may, in some implementations of the present technology, when so configured by the control programs 122, execute a process referred to herein as the supervisor. The role of the supervisor is to supervise the feedback controller 310 of the CLNS system 300 to ensure that it delivers appropriate stimuli to the patient.

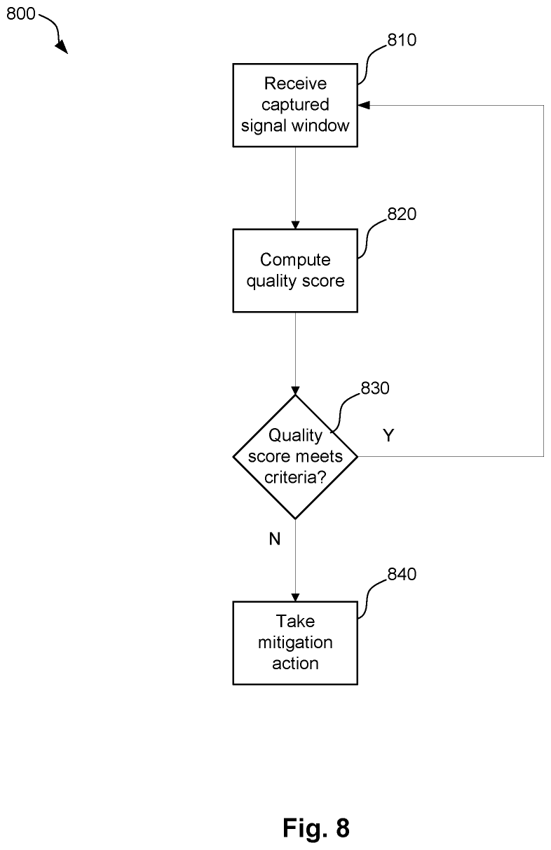

FIG. 8 is a flow chart illustrating a method 800 carried out by the controller 116 as one implementation of the supervisor according to the present technology. The supervisor method 800 starts at step 810, which receives a captured signal window from the measurement circuitry 128. At step 820, the supervisor analyses the captured signal window to compute a quality score indicative of how closely the captured signal window resembles, or how likely that the captured signal window contains, an ECAP. At step 830, the supervisor determines whether the computed quality score meets one or more criteria indicative of satisfactory quality. For example, step 830 may compare the quality score with a threshold to determine whether the quality score exceeds a threshold. If the one or more criteria are met (“Y”), the supervisor returns to step 810 to await the next captured signal window. If the one or more criteria are not met (“N”), the supervisor takes mitigation action at step 840. The method 800 may then, depending on the mitigation action, return to step 810 to await the next captured signal window.

In some implementations, the one or more criteria used at step 830 may be dependent on the type of quality score computed at step 820, and in particular on the range within the quality score must lie. For example, if a criterion is for the quality score to exceed a threshold, and if the quality score must lie in the range [0, 1], the threshold may be 0.5.

In some implementations, a threshold used at step 830 may be a representative value, such as an average, of recent values of the quality score computed at previous iterations of step 820. In such implementations, the supervisor may at step 830 detect a change in circumstances that is affecting the quality of ECAP measurements.

In one implementation of a mitigation action that may be used at step 840, the supervisor may suspend the operation of the feedback controller 310 if the quality score fails to meet the one or more criteria for a significant period, such as ten consecutive iterations of the method 800. In such an implementation, the controller 116 may revert to open-loop stimulation, whereby the stimulus intensity, rather than the target response intensity, is directly controllable by the user of the remote control 720.

In one implementation of a mitigation action that may be used at step 840, the supervisor may select a different measurement electrode configuration.

The following are several implementations of quality score calculation from a captured signal window, as used in step 820 of the supervisor method 800 according to the present technology.

### Noise Departure Detector (NDD)

The NDD is a statistical detector of the presence of an ECAP in a signal window. The operation of the NDD on a signal window is preferably preceded by an “artefact scrubber” which removes artefact from the signal window. One such artefact scrubber is disclosed in International Patent Publication no. WO 2020/124135, the entire contents of which are herein incorporated by reference. The NDD works by detecting a statistically unusual difference from the expected noise present in a signal window. The extent of the difference is indicative of the likelihood of an ECAP in the signal window.

The calibration of an NDD instance corresponding to a measurement electrode configuration (MEC) may be carried out on one or more signal windows captured via that MEC which are known not to contain evoked neural responses. In one implementation, such signal windows are “zero current” signal windows which are captured from intervals during which no stimulus is being applied, and which have preferably been scrubbed for artefact, and may therefore be treated as comprising only noise. The calibration comprises forming estimates of parameters of a predetermined “noise model” (statistical distribution) from the samples in the one or more “zero current” signal windows. In one implementation, the noise model is Gaussian and the parameters are the mean \tilde{μ} and standard deviation \hat{σ} of the samples.

Once calibrated, an NDD instance may be applied to a signal window by counting the number \tilde{k} of outliers in the signal window, i.e. the number of samples in the signal window that depart significantly from the noise model. For a Gaussian noise model, the NDD counts the number \tilde{k} of samples that differ from the mean estimate \tilde{μ} by more than n times the standard deviation estimate \hat{σ}, where n is a small integer. The number \tilde{k} of outliers is compared to the number k of such outliers that would be expected to occur if the signal window consisted solely of noise with mean \tilde{μ} and standard deviation \hat{σ}. The difference between \tilde{k} and k is divided by the number of samples N in the signal window to compute a metric r that quantifies the ratio of outliers present in a signal window relative to the expected ratio of outliers in a signal window that obeys the noise model.

It may be shown that for Gaussian noise model, the NDD may compute the metric r as

$$r = \frac{\tilde{k}}{N} - 2 Φ ( \frac{n}{2} ) \qquad ( 3 )$$

where Φ is the standard normal cumulative distribution function.

A negative or zero value of the metric r indicates a signal window consistent with the noise model, whereas a positive value of r indicates a departure from the noise model.

In one implementation of the NDD, n is set to 3. Smaller values of n make the NDD more sensitive, indicating a departure from noise more readily and increasing the rate of Type I errors (false positives). Conversely, high values for n necessitate large outliers before r will indicate a noise departure, increasing the rate of Type II errors (false negatives).

In one implementation of the NDD, a sigmoid function may be applied to the raw metric r to map the metric r to a quality score Q_{NDD} in the interval [0, 1]:

$${Q}_{NDD} = \frac{1}{1 + exp ( - γ r )} \qquad ( 4 )$$

where γ is a parameter that balances the Type I and Type II errors. In one implementation, γ is set to 50. The NDD quality score Q_{NDD} has a natural interpretation: Q_{NDD}<0.5 corresponds to r≤0 and indicates that the captured signal window is most likely noise. Conversely, Q_{NDD}>0.5 indicates a departure from the noise model in the captured signal window. In implementations of step 820 in which the NDD quality score Q_{NDD} is the computed quality score, a value for the threshold used at step 830 may be 0.5.

In implementations of step 820 in which the NDD quality score Q_{NDD} is the computed quality score, the NDD may be applied to multiple signal windows after they have been averaged together to improve the signal-to-noise ratio. In such implementations, the supervisor method 800 may execute step 810 multiple times to capture and finally average multiple signal windows before proceeding to step 820. In such implementations, the parameters of the noise model may be adjusted depending on the number of signal windows that are averaged. In the Gaussian noise model, the standard deviation \hat{σ} should be divided by the square root of the number of averaged signal windows. In one such implementation, the number of averaged signal windows is eight.

### Normalised Correlation

As mentioned above, the captured signal window may be processed to determine the peak-to-peak amplitude of an ECAP that is presumed to be present in the signal window in accordance with the teachings of International Patent Publication No. WO2015/074121. International Patent Publication No. WO2015/074121 discloses processing the captured signal window using a correlation detector, in which the captured signal window is correlated with a predetermined template referred to as the four-lobe filter. The peak value of the correlation, which may be positive, negative, or zero, is a measure both of the RMS magnitude of the presumed ECAP and of the extent to which the presumed ECAP resembles the template. The sign of the peak correlation value is representative of the phase alignment between the presumed ECAP and the template; a positive sign indicates the two signals are in phase while a negative sign indicates the two signals are in antiphase. If the correlation function exhibits no discernible peak, there is no resemblance between the captured signal window and the template.

This measurement technique is effective to the extent that any ECAP present in the captured signal window morphologically resembles the correlation template. The magnitude of the peak correlation value, normalised by the RMS magnitude of the captured signal window and the correlation template, may therefore be used as an indicator of the resemblance between the captured signal window and the correlation template. Step 820 may therefore compute the quality score as a normalised correlation between the captured signal window and the template by dividing the peak correlation value by the RMS magnitude of the captured signal window and the RMS magnitude of the correlation template.

### Partitioned Correlation

A normalised correlation may return a high value even for captured signal windows that do not resemble the entire correlation template, but contain a non-ECAP-related transient such as a large spike. A more robust quality score may be computed by partitioning the correlation template into multiple (say N) separate portions, each representing a separate feature of an ECAP. In one example, the portions of the template may be portions corresponding to the P1 peak, the N1 peak, and the P2 peak (see FIG. 6).

To compute the partitioned correlation, at each correlation lag, a value is computed for each portion of the template rather than a single value for the template as a whole. The result is N component correlation functions representing the resemblance of the captured signal window to the respective portions of the template over a range of correlation lags. Summing these N component correlation functions together into a single correlation function would reproduce the correlation function representing the resemblance of the captured signal window to the template as a whole. Instead of summing the component correlation functions, they may be combined in such a way that a peak needs to be present in approximately the same location in each component correlation function in order for there to be a peak at that location in the combined correlation function. This is not the case for a sum, in which a single large peak at a given location in one of the component correlation functions (as for example would be produced by a large transient spike) will result in a peak at that location in the summed correlation function. One example of such a combination is a product. Optionally, the component correlation functions may be normalised before combining them. The peak value of the combined correlation function may then be taken as the quality score. The result of such a partitioned correlation is that portions of a captured signal window need to resemble all the respective portions of the correlation template in the proper temporal relation to result in a high quality score.

Such a partitioned correlation method increases the fidelity of the quality score, i.e. its robustness against non-ECAP-resembling transients in the captured signal window that might “leak through” a normalised correlation value resulting from a single correlation against the complete template.

### Regression-Based Methods

In some implementations of step 820, the captured signal window may be modelled as the sum of various components, plus noise. Examples of components are ECAPs, late responses, and artefact. A late response is a myoelectric response to stimulation that is not a propagating action potential, but may be used as a proxy for a neuromuscular (efferent) response (motor fibre activation). The late, or slow, response is so termed because it usually appears later in time than the ECAP after a stimulus.

Each component may be modelled using a parametrised function of time. Examples of component models are described below for the components listed above. Regression is used to determine the parameters of the model of each component in various combinations that best fits the signal window data. Examples of combination models are:
ECAP alone
Artefact alone
ECAP plus artefact

Each fitted combination model may be assigned a goodness-of-fit (GOF) metric that balances the complexity of the combination model with its power to explain the data. The quality metric may be computed from the GOF metrics of the various combination models fitted to the data.

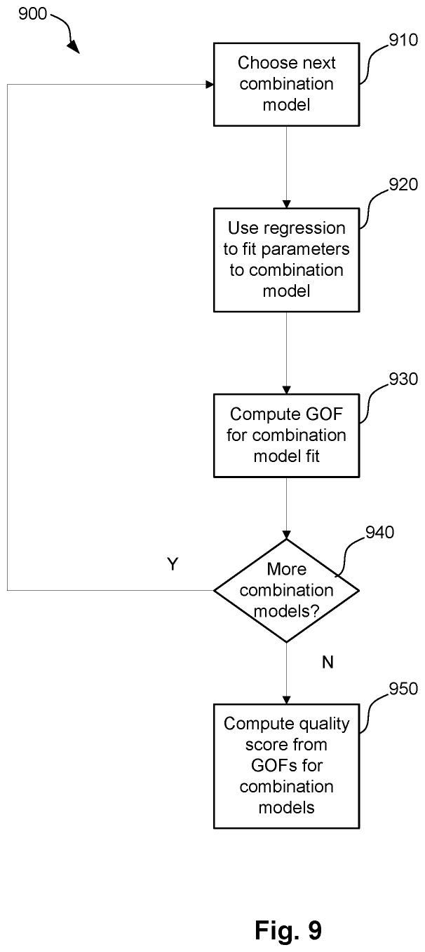

FIG. 9 is a flow chart illustrating a method 900 of computing a quality score using regression and component models, as may be used by the supervisor to implement step 820 of the method 800 according to one implementation of the present technology. The method 900 starts at step 910, which chooses the next combination model containing one or more component models. Step 920 uses regression to jointly fit parameters of the component models making, up the combination model chosen at step 910 to the captured signal window. Step 930 computes a GOF metric indicative of the quality of the combination model fit. An example of a GOF metric for linear regression is the F-test statistic. An example of a GOF metric that may be used for linear or non-linear regression is the Bayesian Information Criterion (BIC).

Step 940 then checks whether there are any more combination models to be chosen. If so (“Y”), the method 900 returns to step 910 to choose another combination model. If not (“N”), step 950 computes the quality score from the GOF metrics for the combination models.

In one implementation, suitable for the three-combination-model example listed above, the quality score may be computed as the difference between the greater of the GOF metric for the (ECAP plus artefact) combination model and the GOF metric for the ECAP-only combination model, and the GOF metric of the artefact-only combination model. In such an implementation, if the quality score is positive, an ECAP is more likely present in the captured signal window than not.

In another implementation, suitable for a result in which the combination model corresponding to the largest GOF metric contains an ECAP, the quality score may be computed as the difference between the largest GOF metric and the GOF metric for the most complex model (meaning the model with the greatest number of parameters).

Component models for ECAPs and artefact will now be described. The above-mentioned International Patent Publication no. WO2020/124135 by the present applicant discloses a single-ended ECAP model e(f,t) as a product of two functions φ(f,t) and ϕ(f,t), each parametrised by a frequency f.

$$e ( f , t ) = φ ( f , t ) ϕ ( f , t ) \qquad ( 5 )$$

where φ(f,t) is a Gamma probability density function:

$$φ ( f , t ) = {( f , t )}^{0.7} exp ( - 1.67 ft ) \qquad ( 6 )$$

and ϕ(f,t) is a piecewise function composed of one period (1/f) of a sine wave of frequency f followed by a decaying exponential function with time constant 1/2πf of such that the derivative is continuous at their boundary:

$$ϕ ( f , t ) = \{ \begin{matrix}0 , t < \frac{0.38}{2 π f} \\ sin 2 π ft - 0.37 , \frac{0.38}{2 π f} < t < \frac{t}{f} \\ 0.63 - exp ( - 2 π f ( t - \frac{1}{f} ) ) , t > \frac{1}{f}\end{matrix} \qquad ( 7 )$$

The parametrised single-ended ECAP model E₀(t), a model of the single-ended ECAP that would be observed at the stimulus electrode (labelled as electrode 0), is a generalised version of the single-ended ECAP e(f,t) that is scaled by a scaling factor κ₀, dilated in time by a dilation parameter v₀, and delayed in time by a delay t₀:

$${E}_{0} ( t ) = {κ}_{0} e ( f , \frac{t - {t}_{0}}{{v}_{0}} ) \qquad ( 8 )$$

The parametrised single-ended ECAP model E₀(t) is referred to as the originating model.

The frequency f may be arbitrarily set to a fixed value, e.g. 1 kHz, without loss of generality as variations in actual frequency from the set value among the measured ECAPs may be handled by the dilation parameter v₀.

A single-ended ECAP Eⱼ(t) arriving at measurement electrode j may be modelled as a scaled version of the originating model E₀(t), where the scaling, dilation, and delay parameters κⱼ, vⱼ, and tⱼ are specific to electrode j:

$${E}_{j} ( t ) = {κ}_{j} e ( f , \frac{t - {t}_{j}}{{v}_{j}} ) \qquad ( 9 )$$

A differential ECAP ΔEⱼₖ(t) measured between recording electrode j and reference electrode k may therefore be modelled as:

$$Δ {E}_{jk} ( t ) = {κ}_{j} e ( f , \frac{t - {t}_{j}}{{v}_{j}} ) - {κ}_{k} e ( f , \frac{t - {t}_{k}}{{v}_{k}} ) \qquad ( 10 )$$

Equation (10) may be fit to a measured differential ECAP ΔEⱼₖ(t) at a measurement electrode pair (j,k) to estimate the parameters κⱼ, vⱼ, and tⱼ, of the single-ended ECAP model at recording electrode j, and the parameters κₖ, vₖ, and tₖ of the single-ended ECAP model at reference electrode k. The parameters of the differential ECAP model of equation (10) are the scaling, dilation, and delay parameters κⱼ, κₖ, vₖ, vₖ, tⱼ, and tₖ.

Non-linear regression may be used to fit the parameters κⱼ, κₖ, vⱼ, vₖ, tⱼ, and tₖ to a captured signal window represented by a vector y of samples.

In one implementation, an artefact model may be derived from a constant phase element (CPE) model of the electrode-tissue interface. This artefact model is described in the above-mentioned International Patent Publication no. WO2020/124135. The CPE artefact model [symbol] _{CPE} comprises five basis functions ϕᵢ (where i=1, . . . , 5) that are defined for a biphasic stimulus waveform as:

$${ϕ}_{1} = s ( t ) - s ( t - pw ) - s ( t - pw - ipg ) + s ( t - 2 \cdot pw - ipg ) \qquad ( 11 )$$

$${ϕ}_{2} = i ( t ) - i ( t - pw ) - i ( t - pw - ipg ) + i ( t - 2 \cdot pw - ipg ) \qquad ( 12 )$$

$${ϕ}_{3} = s ( t - pw - ipg ) - s ( t - 2 \cdot pw - ipg ) \qquad ( 13 )$$

$${ϕ}_{4} = i ( t - pw - ipg ) - i ( t - 2 \cdot pw - ipg ) \qquad ( 14 )$$

$${ϕ}_{5} = 1 \qquad ( 15 )$$

where pw is the pulse width of each phase of the biphasic stimulus, ipg is the inter-phase gap between the two phases, i(t) is the impulse response of a CPE, defined as a decaying exponential:

$$i ( t ) = {t}^{- α} , t \leq 0 \qquad ( 16 )$$

and s(t) is the step response of a CPE, defined as the integral of the impulse response i(t) over all positive time t:

$$s ( t ) = {\int }_{t = 0}^{t = ∞} {t}^{- α} dt = \frac{1}{1 - α} {t}^{- α} + 𝒞 \qquad ( 17 )$$

The parameter α is a constant that depends on the geometry of the electrode-tissue interface, but in one implementation may be set to 0.364.

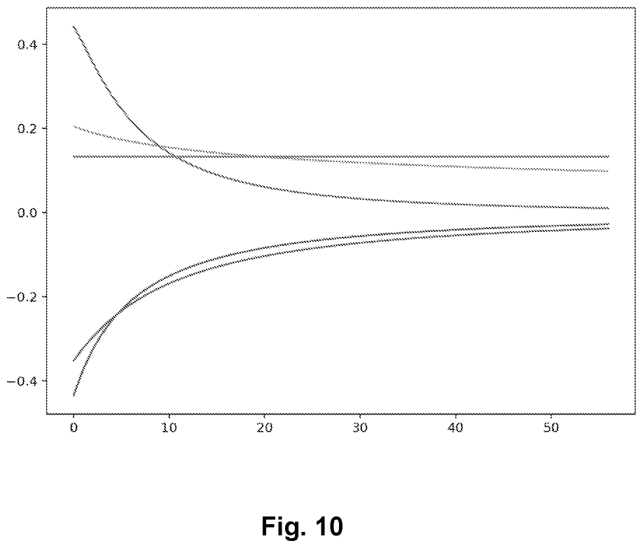

FIG. 10 contains a graph illustrating the five basis functions ϕᵢ in the CPE artefact model [symbol] _{CPE}.

Fitting an artefact model to a captured signal window, represented by a vector y of samples, comprises finding a set of r coefficients {aᵢ: i=1, . . . , r} (e.g. for the CPE model r=5) such that the vector y is most closely approximated (e.g. in a least-squares sense) by a weighted sum of the basis functions ϕᵢ, wherein the weights are the coefficients aᵢ:

$$y \approx {\sum }_{i = 1}^{r} {a}_{i} {ϕ}_{i} \qquad ( 18 )$$

If the basis functions ϕᵢ of a model [symbol] are orthonormal, the least-squares approximation to a signal window y by the model [symbol] may be found by computing inner products of the signal window y with the basis functions ϕᵢ:

$${a}_{i} = 〈 y , {ϕ}_{i} 〉 \qquad ( 19 )$$

However, the CPE basis [symbol] _{CPE} is not orthonormal. The coefficients αᵢ may be found by orthonormalizing the CPE artefact model [symbol] _{CPE}, applying Equation (19) to the orthonormalized basis functions of the CPE artefact model [symbol] _{CPE}, and then transforming the resulting coefficients back to the artefact model [symbol] _{CPE} using conventional linear algebra. This is a form of linear regression in which the fitted coefficients are the model parameters.

Once a combination model has been fitted to the captured signal window, as at step 920, the residuals r may be computed as the difference between the actual samples of the captured signal window and the corresponding samples of the fitted combination model. In the example of an artefact-only combination model in which the artefact is modelled by the CPE basis [symbol] _{CPE}, the residuals are the differences between the weighted sum of the basis functions ϕᵢ with the fitted coefficients {aᵢ: i=1, . . . , r} and the original signal window y:

$$r = y - {\sum }_{i = 1}^{r} {a}_{i} {ϕ}_{i} \qquad ( 20 )$$

In some implementations, the residuals may be compared with a noise model, i.e. a model of the expected noise distribution. One example of a noise model is a zero-mean Gaussian noise model with a standard deviation of 3 μV. In such implementations, the supervisor may check whether the distribution of the residuals of the most complex combination model is significantly different from noise model using a hypothesis test comparing the residual distribution with the noise model, e.g. a Kolmogorov-Smirnoff test. If the distribution of the residuals of the most complex combination model is significantly different from the noise model, there is likely a non-noise component in the captured signal window. The supervisor may in this instance take mitigation action.

In other implementations of the present technology, the supervisor, under certain circumstances, modifies the operation of the feedback controller 310 so that the feedback controller 310 uses the computed quality score as the feedback variable rather the neural response intensity. In one such implementation, the supervisor makes such a modification if the target ECAP amplitude is set by the user (e.g. using the remote controller 720) below a predetermined changeover value, e.g. the perception threshold 410. When the target ECAP amplitude is low, around the ECAP threshold of stimulus intensity, reliable loop operation becomes more difficult as the nature of the ECAP becomes more stochastic, and for a good deal of the time the ECAP will be indistinguishable from noise. The quality score, however, in principle increases smoothly from zero as the stimulus intensity increases and is not subject to such a pronounced “knee” as occurs around the ECAP threshold (see FIG. 4a). This makes the quality score potentially a more reliable value to use as a feedback variable.

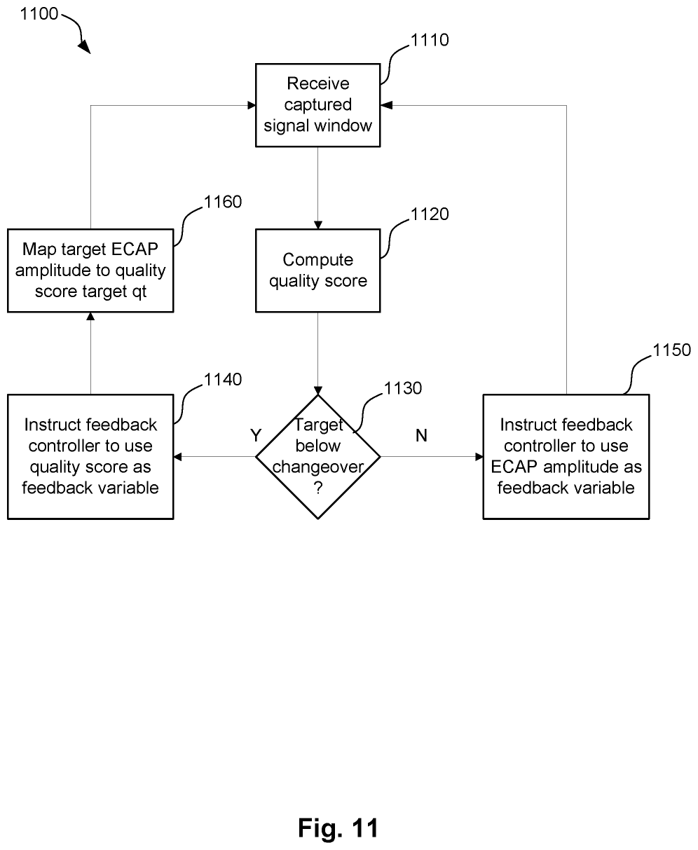

FIG. 11 is a flow chart illustrating a method 1100 carried out by the supervisor according to such implementations of the present technology. The method 1100 is similar to the method 800 in that the steps 1110 and 1120 correspond to the steps 810 and 820 respectively. Step 1130, however, instead of testing the value of the quality score as in step 830, determines whether the current value of the target ECAP amplitude is below the changeover value. If not (“N”), step 1150 instructs the feedback controller 310 to use the ECAP amplitude from the ECAP detector 320 as the feedback variable. After step 1150, the method 1100 returns to step 1110 to await the next captured signal window. If the current value of the target is below the changeover value (“Y”), step 1140 instructs the feedback controller 310 to use the quality score computed at step 1120 as the feedback variable.

At the next step 1160, the supervisor maps the target ECAP amplitude Et set by the remote controller 720 to an appropriate target value qt of the quality score, depending on the typical value Qc of the quality score at the predetermined changeover value Ec of the target ECAP amplitude. In one implementation, the supervisor sets the target value qt of the quality score to be equal to Qc times the ratio of Et to Ec. The mapped target quality score qt is provided to the feedback controller 310 by the target controller 304.

After step 1160, the method 1100 returns to step 1110 to await the next captured signal window.

LABEL LIST

| | |
|---|---|
| stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuitry | 128 |
| ground | 130 |
| electrode array | 150 |
| biphasic stimulus pulse | 160 |
| neural response | 170 |
| nerve | 180 |
| transcutaneous communications channel | 190 |
| external device | 192 |
| closed - loop neural stimulation system | 300 |
| clinical settings controller | 302 |
| target controller | 304 |
| box | 308 |
| box | 309 |
| feedback controller | 310 |
| box | 311 |
| stimulator | 312 |
| element | 313 |
| measurement circuitry | 318 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| target ECAP amplitude | 520 |
| ECAP | 600 |
| neuromodulation system | 700 |
| neuromodulation device | 710 |
| remote control | 720 |
| clinical settings transceiver | 730 |
| clinical interface | 740 |
| charger | 750 |
| method | 800 |
| step | 810 |
| step | 820 |
| step | 830 |
| step | 840 |
| method | 900 |
| step | 910 |
| step | 920 |
| step | 930 |
| step | 940 |
| step | 950 |
| method | 1100 |
| step | 1110 |
| step | 1120 |
| step | 1130 |
| step | 1150 |
| step | 1160 |

### Examples of the Invention

1. A neurostimulation system comprising:
   a neurostimulation device for controllably delivering a neural stimulus, the neurostimulation device comprising:
      a plurality of implantable electrodes including one or more stimulus electrodes and one or more sense electrodes;
      a stimulus source configured to deliver neural stimuli via the one or more stimulus electrodes to a neural pathway of a patient;
      measurement circuitry configured to capture signal windows sensed at the one or more sense electrodes in response to respective neural stimuli; and
      a control unit configured to control the stimulus source to deliver one or more neural stimuli according to a stimulus intensity parameter; and
   a processor configured to:
      detect a neural response evoked by the neural stimuli in the one or more captured signal windows by detecting a difference from a predetermined noise model for the signal windows.
2. The neurostimulation system of example 1, wherein the processor is configured to detect the difference by:
   counting a number of outliers in the or each signal window, wherein an outlier is a sample that departs from the predetermined noise model;
   computing a metric that quantifies a ratio of outliers present in the signal windows relative to an expected ratio of outliers in a signal window that obeys the predetermined noise model; and
   comparing the metric with a threshold to detect the difference.
3. The neurostimulation system of example 2, wherein the processor is further configured to apply a sigmoid function to the metric before comparing the metric with the threshold.
4. The neurostimulation system of any one of examples 2 to 3, wherein the predetermined noise model is a Gaussian model having a mean and a standard deviation.
5. The neurostimulation system of example 4, wherein the processor is further configured to estimate the mean and the standard deviation from signal windows captured without preceding neural stimuli.
6. The neurostimulation system of any one of examples 4 to 5, wherein an outlier is a sample that differs from the mean of the predetermined noise model by more than n times the standard deviation of the predetermined noise model, wherein n is a small integer.
7. The neurostimulation system of any one of examples 1 to 6, wherein the processor is further configured to average a plurality of the captured signal windows before detecting the neural response in the averaged signal windows.
8. The neurostimulation system of any one of examples 1 to 7, wherein the processor is further configured to remove stimulus artefact from each captured signal window before detecting the neural response in the signal windows.
9. The neurostimulation system of any one of examples 1 to 8, wherein the processor is further configured to:
   ramp a value of the stimulus intensity parameter while instructing the control unit to control the stimulus source to deliver the neural stimuli according to the ramping value of the stimulus intensity parameter;
   repeatedly detect a neural response evoked by the neural stimuli in the captured signal windows; and
   record the stimulus intensity parameter at which neural responses are detected in more than 50% of captured signal windows.
10. The neurostimulation system of example 9, wherein:
   the processor is part of an external computing device in communication with the neurostimulation device, and
   the processor is further configured to program, using the recorded value of the stimulus intensity parameter, the neurostimulation device to deliver neural stimulus to the patient.
11. The neurostimulation system of any one of examples 1 to 9, wherein the processor is part of the control unit.
12. The neurostimulation system of any one of examples 1 to 9, wherein the processor is part of an external computing device in communication with the neurostimulation device.
13. An automated method of detecting a neural response evoked by a neural stimulus delivered to a patient, the method comprising:
   capturing a signal window sensed at the one or more sense electrodes in response to the neural stimulus; and
   detecting the neural response evoked by the neural stimulus in the signal window by detecting a difference from a predetermined noise model for the signal window.
14. The method of example 13, wherein detecting the difference comprises:
   counting a number of outliers in the or each signal window, wherein an outlier is a sample that departs from the predetermined noise model;
   computing a metric that quantifies a ratio of outliers present in the signal windows relative to an expected ratio of outliers in a signal window that obeys the predetermined noise model; and
   comparing the metric with a threshold to detect the difference.
15. The method of example 14, further comprising applying a sigmoid function to the metric before comparing the metric with the threshold.
16. The method of any one of examples 14 to 15, wherein the predetermined noise model is a Gaussian model having a mean and a standard deviation.
17. The method of example 16, further comprising estimating the mean and the standard deviation from signal windows captured without preceding neural stimuli.
18. The method of any one of examples 16 to 17, wherein an outlier is a sample that differs from the mean of the predetermined noise model by more than n times the standard deviation of the predetermined noise model, wherein n is a small integer.
19. The method of any one of examples 13 to 18, further comprising averaging a plurality of the captured signal windows before detecting the neural response in the averaged signal windows.
20. The method of any one of examples 13 to 19, further comprising removing stimulus artefact from each captured signal window before detecting the neural response in the signal windows.
21. The method of any one of examples 13 to 20, further comprising:
   ramping a value of a stimulus intensity parameter while delivering the neural stimuli according to the ramping value of the stimulus intensity parameter;
   repeatedly detecting a neural response evoked by the neural stimuli in the captured signal windows; and
   recording the stimulus intensity parameter at which neural responses are detected in more than 50% of captured signal windows.
22. The method of example 21, further comprising programming, using the recorded value of the stimulus intensity parameter, a neurostimulation device to deliver neural stimuli to the patient.

1. An implantable device for controllably delivering neural stimuli, the device comprising:
a stimulus source configured to provide neural stimuli to be delivered via one or more stimulus electrodes of a plurality of electrodes to a neural pathway of a patient in order to evoke a neural response from the neural pathway;
measurement circuitry configured to capture signal windows sensed on the neural pathway via one or more sense electrodes of the plurality of electrodes subsequent to respective neural stimuli; and
a control unit configured to:
control the stimulus source to provide a neural stimulus according to a stimulus intensity parameter;
measure an intensity of an evoked neural response in the captured signal window subsequent to the provided neural stimulus;
compute a feedback variable from the measured intensity of the evoked neural response; and
implement a feedback controller configured to use the computed feedback variable to control the stimulus intensity parameter so as to maintain the feedback variable at a target value;
compute a quality score from the captured signal window;
determine whether the quality score meets one or more criteria indicative of satisfactory quality; and
take mitigation action based on the determining.

2. The implantable device of claim 1, wherein the control unit is configured to compute the quality score by computing a difference between the captured signal window and a predetermined noise model for the captured signal windows.

3. The implantable device of claim 2, wherein the control unit is configured to compute the difference by:
counting a number of outliers in the signal window, wherein an outlier is a sample that departs from the predetermined noise model; and
computing a metric that quantifies a ratio of outliers present in the signal window relative to an expected ratio of outliers in a signal window that obeys the predetermined noise model.

4. The implantable device of claim 3, wherein the control unit is configured to apply a sigmoid function to the metric.

5. The implantable device of claim 3, wherein an outlier is a sample that differs from the mean of the predetermined noise model by more than n times the standard deviation of the predetermined noise model, wherein n is a small integer.

6. The implantable device of claim 2, wherein the predetermined noise model is a Gaussian model having a mean and a standard deviation.

7. The implantable device of claim 6, wherein the control unit is further configured to estimate the mean and the standard deviation from signal windows captured without preceding neural stimuli.

8. The implantable device of claim 2, wherein the control unit is further configured to remove stimulus artefact from the captured signal window before computing the difference.

9. The implantable device of claim 1, wherein the control unit is configured to compute the quality score by:
computing a normalised correlation function representing a resemblance of the captured signal window to a correlation template.

10. The implantable device of claim 1, wherein the control unit is configured to compute the quality score by:
computing a plurality of component correlation functions, each component correlation function representing a resemblance of the captured signal window to a portion of a correlation template; and
combining the component correlation functions into a combined correlation function.

11. The implantable device of claim 10, wherein a peak value of the combined correlation function is the quality score.

12. The implantable device of claim 1, wherein the control unit is configured to compute the quality score by:
fitting, for each of a plurality of combination models, the combination model to the captured signal window;
computing a plurality of goodness-of-fit metrics indicative of the quality of the model fit of the respective combination models to the captured signal window; and
computing the quality score from the plurality goodness-of-fit metrics for the respective combination models.

13. The implantable device of claim 12, wherein each combination model comprises one or more component models.

14. The implantable device of claim 12, wherein computing the quality score comprises computing a difference between a goodness-of-fit metric for a combination model comprising an ECAP component model and an artefact component model, and a goodness-of-fit metric for a combination model comprising an artefact component model alone.

15. The implantable device of claim 12, wherein computing the quality score comprises computing a difference between a largest goodness-of-fit metric of the plurality of the goodness-of-fit metrics, and a goodness-of-fit metric for a most complex of the combination models.

16. The implantable device of claim 1, wherein the control unit is configured to determine whether the quality score meets one or more criteria indicative of satisfactory quality by comparing the quality score with a threshold.

17. The implantable device of claim 1, wherein the control unit is configured to take mitigation action by suspending the operation of the feedback controller.

18. An automated method of controllably delivering neural stimuli to a neural pathway of a patient, the method comprising:
delivering a neural stimulus to the neural pathway of the patient in order to evoke a neural response from the neural pathway, the neural stimulus being delivered according to a stimulus intensity parameter;
capturing a signal window sensed on the neural pathway subsequent to the delivered neural stimulus;
measuring an intensity of a neural response evoked by the delivered neural stimulus in the captured signal window,
computing, from the measured intensity of the evoked neural response, a feedback variable; and
completing a feedback loop by using the computed feedback variable to control the stimulus intensity parameter so as to maintain the feedback variable at a target value; and
computing a quality score from the captured signal window;
determining whether the quality score meets one or more criteria indicative of satisfactory quality; and
taking mitigation action based on the determining.

19. The method of claim 18, wherein computing the quality score comprises computing a difference between the captured signal window and a predetermined noise model for the captured signal windows.

20. The method of claim 18, wherein computing the quality score comprises computing a normalised correlation function representing a resemblance of the captured signal window to a correlation template.

21. The method of claim 18, wherein computing the quality score comprises:
computing a plurality of component correlation functions, each component correlation function representing a resemblance of the captured signal window to a portion of a correlation
template; and combining the component correlation functions into a combined correlation function.

22. The method of claim 18, wherein computing the quality score comprises:
fitting, for each of a plurality of combination models, the combination model to the captured signal window;
computing a plurality of goodness-of-fit metrics indicative of the quality of the model fit of the respective combination models to the captured signal window; and
computing the quality score from the plurality goodness-of-fit metrics for the respective combination models.

23. The method of claim 18, wherein determining whether the quality score meets one or more criteria indicative of satisfactory quality comprises comparing the quality score with a threshold.

24. The method of claim 18, wherein taking mitigation action comprises suspending the operation of the feedback controller.

25. A closed-loop neural stimulation device for controllably delivering neural stimuli, the device comprising:
a feedback controller configured to use one or more controller parameters to control a stimulus intensity parameter so as to maintain a neural response intensity measured from a captured signal window at a target value; and
a processor configured to:
compute a quality score from the captured signal window;
determine whether the quality score meets one or more criteria indicative of satisfactory quality; and
take mitigation action based on the determining.

26. The closed-loop neural stimulation device of claim 25, wherein the processor is configured to compute the quality score by computing a difference between the captured signal window and a predetermined noise model for the captured signal windows.

27. The closed-loop neural stimulation device of claim 25, wherein the processor is configured to compute the quality score by computing a normalised correlation function representing a resemblance of the captured signal window to a correlation template.

28. The closed-loop neural stimulation device of claim 25, wherein the processor is configured to compute the quality score by:
computing a plurality of component correlation functions, each component correlation function representing a resemblance of the captured signal window to a portion of a correlation template; and
combining the component correlation functions into a combined correlation function.

29. The closed-loop neural stimulation device of claim 25, wherein the processor is configured to compute the quality score by:
fitting, for each of a plurality of combination models, the combination model to the captured signal window;
computing a plurality of goodness-of-fit metrics indicative of the quality of the model fit of the respective combination models to the captured signal window; and
computing the quality score from the plurality goodness-of-fit metrics for the respective combination models.

30. A neural stimulation system comprising:
an implantable device for controllably delivering neural stimuli, the device comprising:
a stimulus source configured to provide neural stimuli to be delivered via one or more stimulus electrodes of a plurality of electrodes to a neural pathway of a patient in order to evoke a neural response from the neural pathway; and
measurement circuitry configured to capture signal windows sensed on the neural pathway via one or more sense electrodes of the plurality of electrodes subsequent to respective neural stimuli; and
a control unit configured to control the stimulus source to provide each neural stimulus according to a stimulus intensity parameter;
a processor configured to:
instruct the control unit to control the stimulus source to provide a neural stimulus according to a stimulus intensity parameter;
measure an intensity of an evoked neural response in the captured signal window subsequent to the provided neural stimulus;
compute a feedback variable from the measured intensity of the evoked neural response;
implement a feedback controller configured to use the computed feedback variable to control the stimulus intensity parameter so as to maintain the feedback variable at a target value;
compute a quality score from the captured signal window;
determine whether the quality score meets one or more criteria indicative of satisfactory quality; and
take mitigation action based on the determining.

31. The neural stimulation system of claim 30, wherein the processor is configured to compute the quality score by computing a difference between the captured signal window and a predetermined noise model for the captured signal windows.

32. The neural stimulation system of claim 30, wherein the processor is configured to compute the quality score by computing a normalised correlation function representing a resemblance of the captured signal window to a correlation template.

33. The neural stimulation system of claim 30, wherein the processor is configured to compute the quality score by:
computing a plurality of component correlation functions, each component correlation function representing a resemblance of the captured signal window to a portion of a correlation template; and
combining the component correlation functions into a combined correlation function.

34. The neural stimulation system of claim 30, wherein the processor is configured to compute the quality score by:
fitting, for each of a plurality of combination models, the combination model to the captured signal window;
computing a plurality of goodness-of-fit metrics indicative of the quality of the model fit of the respective combination models to the captured signal window; and
computing the quality score from the plurality goodness-of-fit metrics for the respective combination models.
